(19) United States
(12) Patent Application Publication
(10) Pub. No.: US 2013/0244235 A1
(43) Pub. Date: Sep. 19, 2013

(54) METHODS AND MATERIALS FOR NONINVASIVE DETECTION OF COLORECTAL NEOPLASIA ASSOCIATED WITH INFLAMMATORY BOWEL DISEASE

(71) Applicant: MAYO FOUNDATION FOR MEDICAL EDUCATION AND RESEARCH, Rochester, MN (US)

(72) Inventors: David A. Ahlquist, Rochester, MN (US); William R. Taylor, Lake City, MN (US); Tracy C. Yab, Rochester, MN (US); John B. Kisiel, Rochester, MN (US)

(73) Assignee: Mayo Foundation for Medical Education and Research, Rochester, MN (US)

(21) Appl. No.: 13/773,791

(22) Filed: Feb. 22, 2013

Related U.S. Application Data
(60) Provisional application No. 61/611,310, filed on Mar. 15, 2012.

(51) Int. Cl. C12Q 1/68

(52) U.S. Cl. CPC C12Q 1/6883
USPC 435/6.11

(57) ABSTRACT

The present invention provides methods and materials related to the detection of colorectal neoplasia (CRN) associated with inflammatory bowel disease (IBD). The present invention provides markers specific for colorectal neoplasia associated with inflammatory bowel disease in or associated with a subject's stool sample. In particular, the present invention provides methods and materials for identifying mammals (e.g., humans) having colorectal neoplasia associated with inflammatory bowel disease by detecting the presence and level of indicators of colorectal neoplasia such as, for example, epigenetic alterations (e.g., DNA methylation) (e.g., CpG methylation) (e.g., CpG methylation in coding or regulatory regions of BMP3, NDRG4, vimentin, EYA4) in DNA from a stool sample obtained from the mammal.

### FIELD OF THE INVENTION

The present invention provides methods and materials related to the detection of colorectal neoplasia (CRN) associated with inflammatory bowel disease (IBD). The present invention provides markers specific for colorectal neoplasia associated with inflammatory bowel disease in or associated with a subject's stool sample. In particular, the present invention provides methods and materials for identifying mammals (e.g., humans) having colorectal neoplasia associated with inflammatory bowel disease by detecting the presence and level of indicators of colorectal neoplasia such as, for example, epigenetic alterations (e.g., DNA methylation) (e.g., CpG methylation) (e.g., CpG methylation in coding or regulatory regions of NDRG4, vimentin, EYA4, and/or BMP3) in DNA from a stool sample obtained from the mammal.

### BACKGROUND OF THE INVENTION

Patients with an inflammatory bowel disease (IBD) are at increased risk for colorectal neoplasia (CRN), including colorectal cancer (CRC) (see, e.g., Rosenquist, et al., Lancet 1959, 1:906; MacDougall, Lancet 1964, 2:655; Ekbom A, N Engl J Med 1990; 323:1228; Weedon D D, N Engl J Med 1973; 289:1099; Softley A, Scand J Gastroenterol Suppl 1988; 144:20; Richards M E, Ann Surg 1989; 209:764; Ekbom A, Lancet 1990; 336:357; Jess T, et al., Gastroenterology 2006; 130:1039-46; Howe H L, et al., Cancer 2006; 107:1711-42; each herein incorporated by reference in its entirety). The risk is related to the duration and anatomic extent of the disease. The mortality in patients diagnosed with colorectal cancer in the setting of IBD is higher than for sporadic colorectal cancer (see, e.g., Richards M E, Ann Surg 1989; herein incorporated by reference in its entirety).

Conventional colonoscopic surveillance, however, is insensitive for detection of colorectal neoplasia associated with inflammatory bowel disease. Improved methods for detection of colorectal neoplasia associated with inflammatory bowel disease are needed.

### SUMMARY

Effective and highly sensitive methods for detecting the presence of colorectal neoplasms (e.g., cancer, adenoma (e.g., advanced adenoma)) associated with IBD (IBD-CRN) are urgently needed in clinical settings, as such assays facilitate diagnosis and clinical intervention at an early stage, thereby leading to much improved rates of recovery and lowering of morbidity and mortality in comparison to diagnostic methods that detect later-stage colorectal cancers associated with IBD. During the course of developing some embodiments of the present invention, it was determined that stool DNA methylation markers (e.g., BMP3, NDRG4, vimentin, EYA4) showed high discrimination for detecting IBD-CRN. In particular, it was demonstrated that a stool assay of methylated BMP3, vimentin, EYA4, or NDRG4 highly discriminated IBD-CRN cases from IBD controls.

Accordingly, the present invention provides methods and materials related to the detection of colorectal neoplasia associated with inflammatory bowel disease (IBD-CRN).

The present invention is not limited to particular methods for detecting colorectal neoplasia associated with inflammatory bowel disease (IBD-CRN). In some embodiments, the present invention provides methods and materials for identifying mammals (e.g., humans) having colorectal neoplasia associated with inflammatory bowel disease by detecting the presence and level of indicators of IBD-CRN in DNA from a stool sample obtained from the mammal. The present invention is not limited to the use of particular indicators of IBD-CRN for identifying mammals (e.g., humans) having colorectal neoplasia associated with inflammatory bowel disease.

In some embodiments, the indicator specific for detection of IBD-CRN includes epigenetic alterations (e.g., DNA methylation) (e.g., CpG methylation) (e.g., CpG methylation in coding or regulatory regions of BMP3, NDRG4, vimentin, EYA4) in DNA from a stool sample obtained from the mammal.

In some embodiments, the indicator specific for detection of IBD-CRN is an epigenetic alteration of vimentin. In some embodiments, the indicator specific for detection of IBD-CRN is an epigenetic alteration of BMP3. In some embodiments, the indicator specific for detection of IBD-CRN is an epigenetic alteration of EYA4. In some embodiments, the indicator specific for detection of IBD-CRN is an epigenetic alteration of NDRG4. Indeed, as noted above, experiments conducted during the course of developing embodiments for the present invention showed that stool DNA methylation markers (e.g., BMP3, NDRG4, vimentin, EYA4) showed high discrimination for detecting IBD-CRN. In particular, it was demonstrated that a stool assay of methylated BMP3, vimentin, EYA4, or NDRG4 highly discriminated IBD-CRN cases from IBD controls. Additional indicators specific for detection of IBD-CRN include, but are not limited to, epigenetic aleterations of bmp-4, SFRP2, septin9, ALX4, TFPI2, PIK3CA, and FOXE1.

The present invention is not limited to manner of detecting the presence or level of epigenetic alterations of indicators specific for IBD-CRN. Epigenetic alterations include but are not limited to DNA methylation (e.g., CpG methylation). In some embodiments, the level (e.g., frequency, score) of methylation (e.g., hypermethylation relative to a control, hypomethylation relative to a control) is determined without limitation to the technique used for such determining. Methods of the present invention are not limited to particular epigenetic alterations (e.g., DNA methylation) (e.g., CpG methylation) (e.g., CpG methylation in coding or regulatory regions of BMP3, vimentin, EYA4, and/or NDRG4). In some embodiments, methylation of a CpG island is assessed. In some embodiments, methylation of a CpG island shore is assessed.

The present invention is not limited to a particular manner of detecting and/or characterizing the methylated markers. In some embodiments, methods for detection of IBD-CRN are configured for detecting and characterizing methylation score, methylation frequency, or methylation level of one or more methylated marker specifics for detection of IBD-CRN (e.g., CpG island or CpG shore biomarkers (e.g., BMP3, vimentin, EYA4, NDRG4)).

The present invention is not limited to a particular technique for assessing DNA methylation levels. Techniques used to assess DNA methylation levels include but are not limited to methylation-specific PCR, quantitative methylation-specific PCR, Restriction Landmark Genomic Scanning for Methylation (RLGS-M), comprehensive high-throughput relative methylation (CHARM) analysis (see, e.g., Irizarry et al. (2009) Nature Gen. 178-186; herein incorporated by reference in its entirety), CpG island microarray, methylated DNA immunopreciptiation, methylation-sensitive DNA restriction enzyme analysis, and bisulfite genomic sequencing PCR, methylation-specific PCR, quantitative methylation-specific PCR, methylation-sensitive DNA restriction enzyme analysis, quantitative bisulfite pyrosequencing, and bisulfite genomic sequencing PCR.

The present invention is not limited to particular methods for obtaining methylated markers. In some embodiments, the methods involve obtaining a stool sample from a mammal, extracting DNA from the stool sample such that the integrity of the DNA is substantially similar to the integrity of the DNA in unexcreted stool from the mammal, and detecting the level of indicators specific for detection of IBD-CRN (e.g., BMP3, vimentin, EYA4, NDRG4).

In some embodiments, the indicator specific for detection of IBD-CRN includes mutated nucleic acids in DNA from a stool sample obtained from the mammal. The methods are not limited to particular mutated nucleic acids for detecting the presence of a colorectal neoplasm in a mammal. In some embodiments, the mutation is a single point mutation in a biomarker of interest. In some embodiments, more than one mutation is present in a biomarker of interest. Mutations may be single base pair deletions, substitutions, or additions; or deletions, substitions, additions, rearrangements (e.g., inversions, transversions) of more than one base pair. Methods of the present invention are not limited by particular biomarkers for detecting mutated nucleic acid. Biomarkers include but are not limited to KRAS, APC, melanoma antigen gene, p53, BRAF, BAT26, and PIK3CA and regions associated with such biomarkers. Mutations in one, two, three, four, or four or more nucleic acid polymers may be detected.

Detection of the presence (e.g., level, frequency, score) of single point mutations is not limited by the technique used for such detection. In some embodiments, techniques used for detection of single point mutations include but are not limited to allele-specific PCR, mutant-enriched PCR, digital protein truncation test, direct sequencing, molecular beacons, and BEAMing. In some embodiments, a region (e.g., a mutation cluster region) is surveyed for level of mutations (e.g., mutation score, mutation frequency) (e.g., presence of multiple mutations), without limitation to the technique used to determine the level of mutation. Techniques used to assess mutation levels in, for example, mutation cluster regions include but are not limited to melt curve analysis, temperature gradient gel electrophoresis, and digital melt curve assay. In some preferred embodiments, digital melt curve assay is used.

The methods are not limited to a particular type of mammal In some embodiments, the mammal is a human.

The methods are not limited to a particular type or stage of inflammatory bowel disease. In some embodiments, the IBD is ulcerative colitis or Crohn's disease (proximal or distal) (see, e.g., Baumgart D C, Carding S R (2007) Lancet 369 (9573): 1627-40; Baumgart D C, Sandborn W J (2007) Lancet 369 (9573): 1641-57; Xavier R J, Podolsky D K (2007) Nature 448 (7152):427-34; each herein incorporated by reference in its entirety). In some embodiments, the IBD is collagenous colitis, lymphocytic colitis, ischemic colitis, diversion colitis, Behcet's disease, or indeterminate colitis.

The methods are not limited to a particular type or stage of colorectal neoplasm. In some embodiments, the colorectal neoplasm is premalignant. In some embodiments, the colorectal neoplasm is malignant. In some embodiments, the colorectal neoplasm is colorectal cancer without regard to stage of the cancer (e.g., stage I, II, III, or IV). In some embodiments, the colorectal neoplasm is adenoma, without regard to the size of the adenoma (e.g., greater than 3 cm; less than or equal to 3 cm; greater than 1 cm; less than or equal to 1 cm). In some embodiments, the adenoma is considered to be an advanced adenoma.

In some embodiments wherein a colorectal neoplasm associated with IBD is detected, additional techniques are performed to characterize the colorectal neoplasm (e.g., to characterize the colorectal neoplasm as malignant or premalignant) (e.g., to characterize the colorectal neoplasm within a particular stage of colorectal cancer).

In certain embodiments, the present invention provides kits for detecting the presence of a colorectal neoplasm associated with IBD in a mammal. In some embodiments, such kits include reagents useful, sufficient, or necessary for detecting and/or characterizing one or more indicators specific for a colorectal neoplasm associated with IBD (e.g., vimentin, NDRG4, EYA4). In some embodiments, the kits contain the reagents necessary to detect the presence or level of epigenetic alterations of indicators specific for IBD-CRN (e.g., DNA methylation) (e.g., CpG methylation) (e.g., CpG methylation in coding or regulatory regions of BMP3, vimentin, EYA4, and/or NDRG4) (e.g., methylation of a CpG island) (e.g., methylation of a CpG island shore). In some embodiments, the kits contain the reagents necessary to detect and characterize methylation score, methylation frequency, or methylation level of one or more methylated marker specifics for detection of IBD-CRN (e.g., CpG island or CpG shore biomarkers (e.g., BMP3, vimentin, EYA4, NDRG4)). In some embodiments, the kits contain the reagents necessary to assess DNA methylation levels (e.g., methylation-specific PCR, quantitative methylation-specific PCR, Restriction Landmark Genomic Scanning for Methylation (RLGS-M), comprehensive high-throughput relative methylation (CHARM) analysis (see, e.g., Irizarry et al. (2009) Nature Gen. 178-186; herein incorporated by reference in its entirety), CpG island microarray, methylated DNA immunopreciptiation, methylation-sensitive DNA restriction enzyme analysis, and bisulfite genomic sequencing PCR, methylation-specific PCR, quantitative methylation-specific PCR, methylation-sensitive DNA restriction enzyme analysis, quantitative bisulfite pyrosequencing, and/or bisulfite genomic sequencing PCR). In some embodiments, the kits contain the reagents necessary to detect the presence or level of mutated nucleic acids in DNA specific for IBD-CRN from a stool sample obtained from the mammal (e.g., KRAS, APC, melanoma antigen gene, p53, BRAF, BAT26, and PIK3CA and regions associated with such biomarkers). In some embodiments, the kits contain the ingredients and reagents necessary to obtain and store a stool sample from a subject.

In certain embodiments, the present invention provides methods for monitoring a treatment of IBD-CRN. For example, in some embodiments, the methods may be performed immediately before, during and/or after a treatment to monitor treatment success. In some embodiments, the methods are performed at intervals on disease-free patients to ensure or monitor treatment success.

In certain embodiments, the present invention provides methods for obtaining a subject's risk profile for developing IBD-CRN. In some embodiments, the subject is diagnosed with IBD but not CRN. In some embodiments, such methods involve obtaining a stool sample from a subject (e.g., a human at risk for developing colorectal cancer; a human diagnosed with IBD but not CRN; a human undergoing a routine physical examination), detecting the presence or absence of one or more indicators specific for IBD-CRN (e.g., detecting the presence, absence, or level of markers specific for IBD-CRN in or associated with the stool sample (e.g., methylation level, score or frequency) (e.g., detecting the presence or level of mutated nucleic acids in DNA specific for IBD-CRN from a stool sample obtained from the mammal (e.g., KRAS, APC, melanoma antigen gene, p53, BRAF, BAT26, and PIK3CA and regions associated with such biomarkers) in the stool sample, and generating a risk profile for developing IBD-CRN based upon the detected presence, absence, or level of the indicators specific for IBD-CRN (e.g., BMP3, vimentin, EYA4, NDRG4). In some embodiments, the risk profile indicates a subject's risk for developing IBD-CRN or a subject's risk for re-developing IBD-CRN. In some embodiments, the risk profile indicates a subject to be, for example, a very low, a low, a moderate, a high, and a very high chance of developing or re-developing IBD-CRN. In some embodiments, a health care provider (e.g., an oncologist) will use such a risk profile in determining a course of treatment or intervention (e.g., colonoscopy, watchful waiting, referral to an oncologist, referral to a surgeon, etc.).

In certain embodiments, the present invention provides methods for detecting colorectal neoplasia in a subject having inflammatory bowel disease. The present invention is not limited to particular methods for detecting colorectal neoplasia in a subject having inflammatory bowel disease. For example, in some embodiments, such methods comprise obtaining DNA from an excreted stool sample of a subject (e.g., a human subject diagnosed with inflammatory bowel disease) and determining the level or presence of one or more nucleic acid polymer markers specific for IBD-CRN. In some embodiments, the one or more nucleic acid polymer markers specific for IBD-CRN include markers having altered methylation in the DNA from the excreted stool sample. In some embodiments, the one or more nucleic acid polymer markers having altered methylation are specific for colorectal neoplasia associated with inflammatory bowel disease. In some embodiments, the one or more nucleic acid polymer markers specific for IBD-CRN include mutated nucleic acids from the excreted stool sample (e.g., KRAS, APC, melanoma antigen gene, p53, BRAF, BAT26, and PIK3CA and regions associated with such biomarkers). In some embodiments, the methods further include generating a risk profile based upon the determined level of the one or more nucleic acid polymer markers having altered methylation in the DNA from the excreted stool sample.

The methods are not limited to particular nucleic acid polymer markers having altered methylation specific for colorectal neoplasia associated with inflammatory bowel disease. In some embodiments, the nucleic acid polymers with altered methylation comprise a region selected from the group consisting of a CpG island and a CpG island shore. In some embodiments, the CpG island or shore is present in a coding region or a regulatory region of a gene selected from the group consisting of BMP3, vimentin, NDRG4, and EYA4. In some embodiments, determining of the level of altered methylation of a nucleic acid polymer comprises determining the methylation score of the CpG island or island shore. In some embodiments, the determining of the level of altered methylation of a nucleic acid polymer comprises determining the methylation frequency of the CpG island or island shore. In some embodiments, determining of the level of a nucleic acid polymer with altered methylation is achieved by a technique including, but not limited to, methylation-specific PCR, quantitative methylation-specific PCR, methylation-sensitive DNA restriction enzyme analysis, quantitative bisulfite pyrosequencing, and bisulfite genomic sequencing PCR.

The methods are not limited to a particular type of colorectal neoplasm. In some embodiments, the colorectal neoplasm is premalignant. In some embodiments, the colorectal neoplasm is malignant.

The methods are not limited to a particular type of inflammatory bowel disease. In some embodiments, the inflammatory bowel disease is ulcerative colitis. In some embodiments, the inflammatory bowel disease is Crohn's disease.

In certain embodiments, the present invention provides kits for detecting the presence of a colorectal neoplasm in a mammal having inflammatory bowel disease, the kit comprising reagents useful, sufficient, or necessary for detecting and/or characterizing one or more nucleic acid polymers with altered methylation specific for colorectal neoplasm associated with inflammatory bowel disease from a stool sample, wherein the one or more nucleic acid polymers are selected from the group consisting of BMP3, vimentin, NDRG4, and EYA4. Additional embodiments will be apparent to persons skilled in the relevant art based on the teachings contained herein.

### BRIEF DESCRIPTION OF THE DRAWINGS

FIG. 1 shows gene mutations detected in tissue DNA from Inflammatory Bowel Disease associated cancers (n=25).

FIG. 2 shows Receiver Operating Characteristics Curve for detection of neoplasms by stool assay of methylated A) BMP3, B) Vimentin, C) EYA4 and D) NDRG4 (AUC, area under curve; CRC, colorectal cancer).

FIG. 3 shows distribution of copies of methylated A) BMP3, B) Vimentin, C) EYA4 and D) NDRG4 obtained from case and control stool samples (CRC, colorectal cancer; LGD, low-grade dysplasia; HGD, high-grade dysplasia).

### DEFINITIONS

To facilitate an understanding of the present invention, a number of terms and phrases are defined below.

As used herein, the term “sensitivity” is defined as a statistical measure of performance of an assay (e.g., method, test), calculated by dividing the number of true positives by the sum of the true positives and the false negatives.

As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (or panel of markers) in a positive sample.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition (e.g., disease, disorder), substantially ameliorating clinical symptoms of a condition (e.g., disease, disorder) or substantially preventing the appearance of clinical symptoms of a condition (e.g., disease, disorder).

As used herein, the term “preventing” refers to barring a subject from acquiring a disorder or disease in the first place.

As used herein, the term “CpG island” refers to a genomic DNA region that contains a high percentage of CpG sites relative to the average genomic CpG incidence (per same species, per same individual, or per subpopulation (e.g., strain, ethnic subpopulation, or the like). Various parameters and definitions for CpG islands exist; for example, in some embodiments, CpG islands are defined as having a GC percentage that is greater than 50% and with an observed/expected CpG ratio that is greater than 60% (Gardiner-Garden et al. (1987) J. Mol. Biol. 196:261-282; Baylin et al. (2006) Nat. Rev. Cancer 6:107-116; Irizarry et al. (2009) Nat. Genetics 41:178-186; each herein incorporated by reference in its entirety). In some embodiments, CpG islands may have a GC content >55% and observed CpG/expected CpG of 0.65 (Takai et al. (2007) PNAS 99:3740-3745; herein incorporated by reference in its entirety). Various parameters also exist regarding the length of CpG islands. As used herein, CpG islands may be less than 100 bp; 100-200 bp, 200-300 bp, 300-500 bp, 500-750 bp; 750-1000 bp; 100 or more by in length. In some embodiments, CpG islands show altered methylation patterns relative to controls (e.g., altered methylation in cancer subjects relative to subjects without cancer; tissue-specific altered methylation patterns; altered methylation in stool from subjects with colorectal neoplasia (e.g., colorectal cancer, colorectal adenoma) relative to subjects without colorectal neoplasia). In some embodiments, altered methylation involves hypermethylation. In some embodiments, altered methylation involves hypomethylation.

As used herein, the term “CpG shore” or “CpG island shore” refers to a genomic region external to a CpG island that is or that has potential to have altered methylation patterns (see, e.g., Irizarry et al. (2009) Nat. Genetics 41:178-186; herein incorporated by reference in its entirety). CpG island shores may show altered methylation patterns relative to controls (e.g., altered methylation in cancer subjects relative to subjects without cancer; tissue-specific altered methylation patterns; altered methylation in stool from subjects with colorectal neoplasia (e.g., colorectal cancer, colorectal adenoma) relative to subjects without colorectal neoplasia). In some embodiments, altered methylation involves hypermethylation. In some embodiments, altered methylation involves hypomethylation. CpG island shores may be located in various regions relative to CpG islands (see, e.g., Irizarry et al. (2009) Nat. Genetics 41; 178-186; herein incorporated by reference in its entirety). Accordingly, in some embodiments, CpG island shores are located less than 100 bp; 100-250 bp; 250-500 bp; 500-1000 bp; 1000-1500 bp; 1500-2000 bp; 2000-3000 bp; 3000 bp or more away from a CpG island.

As used herein, the term “inflammatory bowel disesase (IBD),” or similar term, refers to a disorder or disease characterized by inflammatory activity in the GI tract. Examples of IBDs include, without limitation, Crohn's disease (both distal and proximal), ulcerative colitis, indeterminate colitis, microscopic colitis, collagenous colitis, idiopathic inflammation of the small and/or proximal intestine and IBD-related diarrhea.

As used herein, the term “colorectal cancer” is meant to include the well-accepted medical definition that defines colorectal cancer as a medical condition characterized by cancer of cells of the intestinal tract below the small intestine (e.g., the large intestine (colon), including the cecum, ascending colon, transverse colon, descending colon, and sigmoid colon, and rectum). Additionally, as used herein, the term “colorectal cancer” is meant to further include medical conditions which are characterized by cancer of cells of the duodenum and small intestine (jejunum and ileum).

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body. As used herein, the term “metastasized colorectal cancer cells” is meant to refer to colorectal cancer cells which have metastasized; colorectal cancer cells localized in a part of the body other than the duodenum, small intestine (jejunum and ileum), large intestine (colon), including the cecum, ascending colon, transverse colon, descending colon, and sigmoid colon, and rectum.

As used herein, “an individual is suspected of being susceptible to metastasized colorectal cancer” is meant to refer to an individual who is at an above-average risk of developing metastasized colorectal cancer. Examples of individuals at a particular risk of developing metastasized colorectal cancer are those whose family medical history indicates above average incidence of colorectal cancer among family members and/or those who have already developed colorectal cancer and have been effectively treated who therefore face a risk of relapse and recurrence. Other factors which may contribute to an above-average risk of developing metastasized colorectal cancer which would thereby lead to the classification of an individual as being suspected of being susceptible to metastasized colorectal cancer may be based upon an individual's specific genetic, medical and/or behavioral background and characteristics.

The term “neoplasm” as used herein refers to any new and abnormal growth of tissue. Thus, a neoplasm can be a premalignant neoplasm or a malignant neoplasm. The term “neoplasm-specific marker” refers to any biological material that can be used to indicate the presence of a neoplasm. Examples of biological materials include, without limitation, nucleic acids, polypeptides, carbohydrates, fatty acids, cellular components (e.g., cell membranes and mitochondria), and whole cells. The term “colorectal neoplasm (CRN)” as used herein refers to any new and abnormal growth of colorectal tissue. The term “colorectal neoplasm-specific marker” refers to any biological material that can be used to indicate the presence of a colorectal neoplasm (e.g., a premalignant colorectal neoplasm; a malignant colorectal neoplasm). The term “colorectal neoplasm-specific marker associated with inflammatory bowel disease” refers to any biological material that can be used to indicate the presence of a colorectal neoplasm (e.g., a premalignant colorectal neoplasm; a malignant colorectal neoplasm) associated with inflammatory bowel disease (IBD-CRN). Examples of IBD-CRN specific markers include, but are not limited to, hypermethlated markers (e.g., vimentin, EYA4, and NDRG4).

As used herein, the term “adenoma” refers to a benign tumor of glandular origin. Although these growths are benign, over time they may progress to become malignant. As used herein the term “colorectal adenoma” refers to a benign colorectal tumor in which the cells form recognizable glandular structures or in which the cells are clearly derived from glandular epithelium.

As used herein, the term “amplicon” refers to a nucleic acid generated using primer pairs. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specfic PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et1 al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N-6-methyladenosine, aziridinylcytosine, pseudoisocytosine, 5-(carboxyhydroxyl-methyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem. Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

### DETAILED DESCRIPTION OF THE INVENTION

Patients with inflammatory bowel disease (IBD) are at increased risk of colorectal neoplasia (CRN), including colorectal cancer (CRC) (see, e.g., Jess T, et al., Gastroenterology 2006; 130:1039-46; Howe H L, et al., Cancer 2006; 107:1711-42; each herein incorporated by reference in its entirety). Factors known to increase CRC risk in IBD include, for example, duration and extent of chronic ulcerative colitis (CUC) or Crohn's colitis (CD), presence of primary sclerosing cholangitis (PSC), degree of histological activity, and family history of CRC (see, e.g., Itzkowitz S H, Gastroenterology 2004; 126:1634-48; Cairns S R, et al., Gut 2010; 59:666-89; Colonoscopic Surveillance for Prevention of Colorectal Cancer in People with Ulcerative Colitis, Crohn's Disease or Adenomas. National Institute for Health and Clinical Excellence (UK), 2011; each herein incorporated by reference in its entirety). To reduce CRC risk, patients with IBD undergo surveillance colonoscopy to detect early CRN (dysplasia and cancer).

Surveillance in IBD currently involves performing periodic colonoscopies, taking multiple random biopsies to detect occult dysplasia (see, e.g., Farraye F A, et al., Gastroenterology 2010; 138:746-74, 774 e1-4; quiz e12-3; herein incorporated by reference in its entirety). Limitations of this approach include under-sampling with undirected biopsies, an unknown ideal frequency, and lack of evidence for effectiveness (see, e.g., Karlen P, et al., Gut 1998; 42:711-4; Loftus E V, J Clin Gastroenterol 2003; 36:S79-83; discussion S94-6; each herein incorporated by reference in its entirety). Some centers use image-enhancing techniques such as chromoendoscopy for surveillance. This has the advantage of identifying more dysplastic lesions than random biopsies (see, e.g., Subramanian V, Alimentary Pharmacology & Therapeutics 2011; 33:304-12; herein incorporated by reference in its entirety), but requires special training, and sometimes extended endoscopy time. Regardless of the surveillance technique, CRN may be missed despite surveillance, in large part due, for example, to irregularities of the colonic mucosa from chronic inflammation (see, e.g., Connell W R, Gastroenterology 1994; 107:934-44; Lim C H, Gut 2003; 52:1127-32; each herein incorporated by reference in its entirety).

Stool assay of exfoliated molecular markers represents a noninvasive approach that could serve as an adjunct to colonoscopy (see, e.g., Imperiale T F, N Engl J Med 2004; 351:2704-14; Osborn N K, Gastroenterology 2005; 128:192-206; each herein incorporated by reference in its entirety). Indeed, stool DNA testing has recently been incorporated into practice guidelines for average-risk general population screening of sporadic CRC (see, e.g., Levin B, Gastroenterology 2008; 134:1570-95; Rex D K, Am J Gastroenterol 2009; 104:739-50; each herein incorporated by reference in its entirety) and next generation assay methods have yielded high detection rates for both CRC and precancers (see, e.g., Ahlquist D A, Gastroenterology 2012; 142:248-56; herein incorporated by reference in its entirety). Stool DNA testing has not been explored in the IBD population.

Numerous IBD-CRN tissue studies have evaluated candidate markers including acquired mutations in p53 (see, e.g., Taylor H W, Br J Surg 1993; 80:442-4; Lashner B A, Am J. Gastroenterol 1999; 94:456-62; each herein incorporated by reference in its entirety), APC (see, e.g., Odze R D, Am J Surg Pathol 2000; 24:1209-16; herein incorporated by reference in its entirety), K-ras (see, e.g., Bell S M, Br J Cancer 1991; 64:174-8; Holzmann K, Int J Cancer 1998; 76:1-6; Hirota Y, Oncol Rep 2000; 7:233-9; each herein incorporated by reference in its entirety), and BRAF (see, e.g., Aust D E, Int J Cancer 2005; 115:673-7; herein incorporated by reference in its entirety) as well as aberrant methylation in EYA4 (see, e.g., Osborn N K, Clin Gastroenterol Hepatol 2006; 4:212-8; herein incorporated by reference in its entirety), ER, p16, MYOD, P14, E-cadherin, RUNX3, MINT1 and COX-2 (see, e.g., Issa J-PJ, Cancer Res 2001; 61:3573-3577; Sato F, Cancer Res 2002; 62:6820-2; Wheeler J M, Gut 2001; 48:367-71; Garrity-Park M M, Am J Gastroenterol 2010; 105:1610-9; Watanabe T, International journal of oncology 2011; 38:201-7; each herein incorporated by reference in its entirety). Several genes, such as BMP3, vimentin (VIM) (see, e.g. Zou H, Cancer Epidemiol Biomarkers Prey 2007; 16:2686-96; herein incorporated by reference in its entirety), septin 9 (see, e.g., Grutzmann R, PLoS ONE 2008; 3:e3759; herein incorporated by reference in its entirety), and NDRG4 (see, e.g., Ahlquist D A, Gastroenterology 2012; 142:248-56; herein incorporated by reference in its entirety) are selectively methylated in sporadic CRC but have not been investigated in IBD.

Experiments conducted during the course of developing embodiments for the present invention assessed the discriminant value of the mutation markers p53, APC, BRAF, K-ras and PIK3CA and the methylation markers VIM, BMP3, EYA4 and septin 9 for detection of IBD-CRN based on DNA extracted from well-characterized tissue specimens. In addition, such experiments prospectively assessed the feasibility of stool DNA testing (using the most discriminant tissue markers) for the detection of premalignant and malignant IBD-CRN. It was determined that mutations on P53, APC, KRAS, BRAF or PIK3CA genes were insufficiently informative, but several aberrantly methylated genes (vimentin, EYA4, BMP3, NDRG4) were highly discriminant for detecting colorectal neoplasia associated with inflammatory bowel disease. For example, it was determined that individual stool assay of BMP3, vimentin, EYA4, and NDRG4 markers showed high discrimination with respective areas under the ROC curve of 0.91, 0.91, 0.85, and 0.84 for total IBD-CRN and of 0.97, 0.97, 0.95, and 0.94 for cancer. At a specificity of 91%, stool assay of BMP3 alone detected 70% of dysplasia (95% CI 35-91%) and 100% of cancers (95% CI 63-100%). Such experiments demonstrate feasibility for the noninvasive detection of IBD-CRN by stool DNA testing.

Accordingly, the present invention provides methods and materials related to the detection of colorectal neoplasia (CRN) associated with inflammatory bowel disease (IBD). The present invention provides markers specific for colorectal neoplasia associated with inflammatory bowel disease in or associated with a subject's stool sample. In particular, the present invention provides methods and materials for identifying mammals (e.g., humans) having colorectal neoplasia associated with inflammatory bowel disease by detecting the presence and level of indicators of colorectal neoplasia such as, for example, epigenetic alterations (e.g., DNA methylation) (e.g., CpG methylation) (e.g., CpG methylation in coding or regulatory regions of BMP3, NDRG4, vimentin, EYA4) in DNA from a stool sample obtained from the mammal.

While the present invention exemplifies particular markers specific for detecting colorectal neoplasia associated with inflammatory bowel disease (IBD-CRN), any marker that is correlated with the presence or absence of IBD-CRN may be used. A marker, as used herein, includes, for example, nucleic acid(s) whose production or mutation or lack of production is characteristic of a IBD-CRN. Depending on the particular set of markers employed in a given analysis, the statistical analysis will vary. For example, where a particular combination of markers is highly specific for IBD-CRN, the statistical significance of a positive result will be high. It may be, however, that such specificity is achieved at the cost of sensitivity (e.g., a negative result may occur even in the presence of IBD-CRN). By the same token, a different combination may be very sensitive (e.g., few false negatives, but has a lower specificity).

Particular combinations of markers may be used that show optimal function with different ethnic groups or sex, different geographic distributions, different stages of disease, different degrees of specificity or different degrees of sensitivity. Particular combinations may also be developed which are particularly sensitive to the effect of therapeutic regimens on disease progression. Subjects may be monitored after a therapy and/or course of action to determine the effectiveness of that specific therapy and/or course of action.

The methods of the present invention are not limited to particular indicators of IBD-CRN.

In some embodiments, indicators of IBD-CRN include, for example, epigenetic alterations. Epigenetic alterations include but are not limited to DNA methylation (e.g., CpG methylation). In some embodiments, the level (e.g., frequency, score) of methylation (e.g., hypermethylation relative to a control, hypomethylation relative to a control) is determined without limitation to the technique used for such determining. Methods of the present invention are not limited to particular epigenetic alterations (e.g., DNA methylation) (e.g., CpG methylation) (e.g., CpG methylation in coding or regulatory regions of BMP3, vimentin, EYA4, NDRG4). Altered methylation may occur in, for example, CpG islands; CpG island shores; or regions other than CpG islands or CpG island shores. Indeed, as noted above, experiments conducted during the course of developing embodiments for the present invention showed that stool DNA methylation markers (e.g., BMP3, NDRG4, vimentin, EYA4) showed high discrimination for detecting IBD-CRN. In particular, it was demonstrated that a stool assay of methylated BMP3, vimentin, EYA4, or NDRG4 highly discriminated IBD-CRN cases from IBD controls. Additional indicators specific for detection of IBD-CRN include, but are not limited to, epigenetic aleterations of bmp-4, SFRP2, septin9, ALX4, TFPI2, PIK3CA, and FOXE1.

In certain embodiments, methods, kits, and systems of the present invention involve determination of methylation state of a locus of interest (e.g., in human DNA) (e.g., in human DNA extracted from a stool sample). Any appropriate method can be used to determine whether a particular DNA is hypermethylated or hypomethylated. Standard PCR techniques, for example, can be used to determine which residues are methylated, since unmethylated cytosines converted to uracil following a bisulfite reaction and are replaced by thymidine residues during PCR. PCR reactions can contain, for example, 10 μL of captured DNA that either has or has not been treated with sodium bisulfite, 1× PCR buffer, 0.2 mM dNTPs, 0.5 μM sequence specific primers (e.g., primers flanking a CpG island or CpG shore within the captured DNA), and 5 units DNA polymerase (e.g., Amplitaq DNA polymerase from Applied Biosystems, Foster City, Calif.) in a total volume of 50 μl. A typical PCR protocol can include, for example, an initial denaturation step at 94° C. for 5 min, 40 amplification cycles consisting of 1 minute at 94° C., 1 minute at 60° C., and 1 minute at 72° C., and a final extension step at 72° C. for 5 minutes.

To analyze which residues within a captured DNA are methylated, the sequences of PCR products corresponding to samples treated with and without sodium bisulfite can be compared. The sequence from the untreated DNA will reveal the positions of all cytosine residues within the PCR product. Cytosines that were unmethylated will be converted to thymidine residues in the sequence of the bisulfite-treated DNA, while residues that were methylated will be unaffected by bisulfite treatment.

Similarly, in some embodiments, methods of the present invention involve the determination (e.g., assessment, ascertaining, quantitation) of methylation level of an indicator of IBD-CRN (e.g., the mutation level of a CpG island or CpG shore in the coding or regulatory region of a gene locus) in a sample (e.g., a DNA sample extracted from stool). A skilled artisan understands that an increased, decreased, informative, or otherwise distinguishably different methylation level is articulated with respect to a reference (e.g., a reference level, a control level, a threshold level, or the like). For example, the term “elevated methylation” as used herein with respect to the methylation status (e.g., CpG DNA methylation) of a gene locus (e.g., BMP3, vimentin, EYA4, NDRG4) is any methylation level that is above a median methylation level in a stool sample from a random population of mammals (e.g., a random population of 10, 20, 30, 40, 50, 100, or 500 mammals) that do not have IBD-CRN. Elevated levels of methylation can be any level provided that the level is greater than a corresponding reference level. For example, an elevated methylation level of a locus of interest (e.g., BMP3, vimentin, EYA4, NDRG4) methylation can be 0.5, 1, 2, 3, 4, 5, 6, 7, 8, 9, 10, or more fold greater than the reference level methylation observed in a normal stool sample. It is noted that a reference level can be any amount. The term “elevated methylation score” as used herein with respect to detected methylation events in a matrix panel of particular nucleic acid markers is any methylation score that is above a median methylation score in a stool sample from a random population of mammals (e.g., a random population of 10, 20, 30, 40, 50, 100, or 500 mammals) that do not have IBD-CRN. An elevated methylation score in a matrix panel of particular nucleic acid markers can be any score provided that the score is greater than a corresponding reference score. For example, an elevated score of methylation in a locus of interest (e.g., BMP3, vimentin, EYA4, NDRG4) can be 0.5, 1, 2, 3, 4, 5, 6, 7, 8, 9, 10, or more fold greater than the reference methylation score observed in a normal stool sample. It is noted that a reference score can be any amount.

In some embodiments, the indicator specific for detection of IBD-CRN includes mutated nucleic acids in DNA from a stool sample obtained from the mammal. The methods are not limited to particular mutated nucleic acids for detecting the presence of a colorectal neoplasm in a mammal. In some embodiments, the mutation is a single point mutation in a biomarker of interest. In some embodiments, more than one mutation is present in a biomarker of interest. Mutations may be single base pair deletions, substitutions, or additions; or deletions, substitions, additions, rearrangements (e.g., inversions, transversions) of more than one base pair. Methods of the present invention are not limited by particular biomarkers for detecting mutated nucleic acid. Biomarkers include but are not limited to KRAS, APC, melanoma antigen gene, p53, BRAF, BAT26, and PIK3CA and regions associated with such biomarkers. Mutations in one, two, three, four, or four or more nucleic acid polymers may be detected.

Detection of the presence (e.g., level, frequency, score) of single point mutations is not limited by the technique used for such detection. In some embodiments, techniques used for detection of single point mutations include but are not limited to allele-specific PCR, mutant-enriched PCR, digital protein truncation test, direct sequencing, molecular beacons, and BEAMing. In some embodiments, a region (e.g., a mutation cluster region) is surveyed for level of mutations (e.g., mutation score, mutation frequency) (e.g., presence of multiple mutations), without limitation to the technique used to determine the level of mutation. Techniques used to assess mutation levels in, for example, mutation cluster regions include but are not limited to melt curve analysis, temperature gradient gel electrophoresis, and digital melt curve assay. In some preferred embodiments, digital melt curve assay is used.

The methods are not limited to a particular type of mammal. In some embodiments, the mammal is a human.

The methods are not limited to a particular type or stage of inflammatory bowel disease. In some embodiments, the IBD is ulcerative colitis or Crohn's disease (proximal or distal) (see, e.g., Baumgart D C, Carding S R (2007) Lancet 369 (9573): 1627-40; Baumgart D C, Sandborn W J (2007) Lancet 369 (9573): 1641-57; Xavier R J, Podolsky D K (2007) Nature 448 (7152):427-34; each herein incorporated by reference in its entirety). In some embodiments, the IBD is collagenous colitis, lymphocytic colitis, ischemic colitis, diversion colitis, Behcet's disease, or indeterminate colitis.

The methods are not limited to a particular type or stage of colorectal neoplasm. In some embodiments, the colorectal neoplasm is premalignant. In some embodiments, the colorectal neoplasm is malignant. In some embodiments, the colorectal neoplasm is colorectal cancer without regard to stage of the cancer (e.g., stage I, II, III, or IV). In some embodiments, the colorectal neoplasm is adenoma, without regard to the size of the adenoma (e.g., greater than 3 cm; less than or equal to 3 cm; greater than 1 cm; less than or equal to 1 cm). In some embodiments, the adenoma is considered to be an advanced adenoma.

The present invention also provides methods and materials to assist medical or research professionals in determining whether or not a mammal has IBD-CRN. Medical professionals can be, for example, doctors, nurses, medical laboratory technologists, and pharmacists. Research professionals can be, for example, principal investigators, research technicians, postdoctoral trainees, and graduate students. A professional can be assisted by (1) detecting and/or characterizing one or more indicators specific for a colorectal neoplasm associated with IBD (e.g., BMP3, vimentin, NDRG4, EYA4), and (2) communicating such information to that professional, for example. In some cases, a professional can be assisted by (1) determining the methylation status of genes such as BMP3, vimentin, NDRG4, and/or EYA4, and (2) communicating information about the methylation status of particular genes to the professional.

After the level (score, frequency) of particular markers in a stool sample is reported, a medical professional can take one or more actions that can affect patient care. For example, a medical professional can record the results in a patient's medical record. In some cases, a medical professional can record a diagnosis of a colorectal neoplasia associated with inflammatory bowel disorder, or otherwise transform the patient's medical record, to reflect the patient's medical condition. In some cases, a medical professional can review and evaluate a patient's entire medical record, and assess multiple treatment strategies, for clinical intervention of a patient's condition. In some cases, a medical professional can record a prediction of tumor occurrance with the reported indicators. In some cases, a medical professional can review and evaluate a patient's entire medical record and assess multiple treatment strategies, for clinical intervention of a patient's condition. In some cases, a colonoscopy may be appropriate at this point.

A medical professional can initiate or modify treatment of an inflammatory bowel disease (e.g., Crohn's disease; ulcerative colitis) after determining it to be associasted with colorectal neoplasia. In some cases, a medical professional can compare previous reports and the recently communicated level (score, frequency) of markers, and recommend a change in therapy. In some cases, a medical professional can enroll a patient in a clinical trial for novel therapeutic intervention of colorectal neoplasm. In some cases, a medical professional can elect waiting to begin therapy until the patient's symptoms require clinical intervention.

A medical professional can communicate the assay results to a patient or a patient's family. In some cases, a medical professional can provide a patient and/or a patient's family with information regarding colorectal neoplasia associated with inflammatory bowel disease, including treatment options, prognosis, and referrals to specialists, e.g., oncologists and/or radiologists. In some cases, a medical professional can provide a copy of a patient's medical records to communicate assay results to a specialist. A research professional can apply information regarding a subject's assay results to advance colorectal neoplasm research and/or inflammatory bowel disease research. For example, a researcher can compile data on the assay results, with information regarding the efficacy of a drug for treatment of colorectal neoplasia to identify an effective treatment. In some cases, a research professional can obtain assay results to evaluate a subject's enrollment, or continued participation in a research study or clinical trial. In some cases, a research professional can classify the severity of a subject's condition, based on assay results. In some cases, a research professional can communicate a subject's assay results to a medical professional. In some cases, a research professional can refer a subject to a medical professional for clinical assessment of colorectal neoplasia associated with inflammotry bowel disease, and treatment thereof. Any appropriate method can be used to communicate information to another person (e.g., a professional). For example, information can be given directly or indirectly to a professional. For example, a laboratory technician can input the methylation results into a computer-based record. In some cases, information is communicated by making a physical alteration to medical or research records. For example, a medical professional can make a permanent notation or flag a medical record for communicating a diagnosis to other medical professionals reviewing the record. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

It is noted that a single stool sample can be analyzed for one marker specific for IBD-CRN (e.g., epigenetic alterations associated with BMP3, vimentin, EYA4 or NDRG4) (e.g., mutated nucleic acid associated with KRAS, APC, melanoma antigen gene, p53, BRAF, BAT26, and PIK3CA and regions associated with such biomarkers) or for multiple markers specific for IBD-CRN. In preferred embodiments, a single stool sample is analyzed for multiple multiple markers specific for IBD-CRN. In addition, multiple stool samples can be collected for a single mammal and analyzed as described herein. Indeed, U.S. Pat. Nos. 5,670,325, 5,741,650, 5,928,870, 5,952,178, and 6,020,137, each herein incorporated by reference in their entireties, for example, describe various methods that can be used to prepare and analyze stool samples. In some embodiments, the stool sample undergoes one or more preprocessing steps before being split into portions. In some embodiments, the stool sample is treated, handled, or preserved in a manner that promotes DNA integrity and/or inhibits DNA degradation (e.g., through use of storage buffers with stabilizing agents (e.g., chelating agents, DNase inhibitors) or handling or processing techniques that promote DNA integrity (e.g., immediate processing or storage at low temperature (e.g., −80 degrees C.)).

The present invention is not limited to a particular manner of detecting nucleic acid markers specific for IBD-CRN from a stool sample. In some embodiments, nucleic acid is amplified. Generally, nucleic acid used as template for amplification is isolated from cells contained in the biological sample according to standard methodologies (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). Pairs of primers that selectively hybridize to genes corresponding to specific markers are contacted with the isolated nucleic acid under conditions that permit selective hybridization. Once hybridized, the nucleic acid primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In some applications, the detection may be performed by visual means. Alternatively, the detection may involve indirect identification of the product via chemiluminescence, radioactive scintigraphy of incorporated radio label or fluorescent label or even via a system using electrical or thermal impulse signals. Generally, the foregoing process is conducted at least twice on a given sample using at least two different primer pairs specific for two different specific markers. Following detection, in some embodiments, the results seen in a given subject are compared with a statistically significant reference group of subjects diagnosed as not having colorectal neoplasm associated with inflammatory bowel disease.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

In most cases, it will be preferable to synthesize desired oligonucleotides. Suitable primers can be synthesized using commercial synthesizers using methods well known to those of ordinary skill in the art. Where double-stranded primers are desired, synthesis of complementary primers is performed separately and the primers mixed under conditions permitting their hybridization.

Selection of primers is based on a variety of different factors, depending on the method of amplification and the specific marker involved. For example, the choice of primer will determine the specificity of the amplification reaction. The primer needs to be sufficiently long to specifically hybridize to the marker nucleic acid and allow synthesis of amplification products in the presence of the polymerization agent and under appropriate temperature conditions. Shorter primer molecules generally require cooler temperatures to form sufficiently stable hybrid complexes with the marker nucleic acid and may be more susceptible to non-specific hybridization and amplification.

Primer sequences do not need to correspond exactly to the specific marker sequence. Non-complementary nucleotide fragments may be attached to the 5′ end of the primer with the remainder of the primer sequence being complementary to the template. Alternatively, non-complementary bases can be interspersed into the primer, provided that the primer sequence has sufficient complementarily, in particular at the 3′ end, with the template for annealing to occur and allow synthesis of a complementary DNA strand.

In some embodiments, primers may be designed to hybridize to specific regions of the marker nucleic acid sequence. For example, GC rich regions are favored as they form stronger hybridization complexes than AT rich regions. In another example, primers are designed, solely, to hybridize to a pair of exon sequences, with at least one intron in between. This allows for the activity of a marker gene to be detected as opposed to its presence by minimizing background amplification of the genomic sequences and readily distinguishes the target amplification by size. Primers also may be designed to amplify a particular segment of marker nucleic acid that encodes restriction sites. A restriction site in the final amplification product would enable digestion at that particular site by the relevant restriction enzyme to produce two products of a specific size. Any restriction enzyme may be utilized in this aspect. This added refinement to the amplification process may be necessary when amplifying a marker nucleic acid sequence with close sequence similarity to other nucleic acids. Alternatively, it may be used as an added confirmation of the specificity of the amplification product.

A number of template dependent processes are available to amplify the marker sequences present in a given template sample. One of the best known amplification methods is the polymerase chain reaction (PCR) (see, e.g., U.S. Pat. Nos. 4,683,195, 4,683,202, 4,800,159, and Innis et al., PCR Protocols, Academic Press, Inc., San Diego, Calif. (1990); each incorporated herein by reference in their entireties). Briefly, in PCR, two primer sequences are prepared which are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

The present invention is not limited to a particular PCR technique. Examples of PCR include, but are not limited to, standard PCR, allele-specific PCR, assembly PCR, asymmetric PCR, digital PCR, hot-start PCR, intersequence-specfic PCR, inverse PCR, ligation-mediated PCR, methylation-specific PCR, miniprimer PCR, multiplex ligation-dependent probe amplification, nested PCR, overlap-extension PCR, real-time PCR, reverse transcription PCR, solid phase PCR, thermal asymmetric interlaced PCR, and Touchdown PCR. Other related amplification methods include TMA, 3SR, NASBA, TAS, and helicase-dependent amplification.

Another method for amplification is the ligase chain reaction (“LCR”) (see, e.g., U.S. Pat. Nos. 4,883,750 and 5,494,810; herein incorporated by reference in its entirety). In LCR, two complementary probe pairs are prepared, and in the presence of the marker sequence, each pair will bind to opposite complementary strands of the marker such that they abut. In the presence of a ligase, the two probe pairs will link to form a single unit. By temperature cycling, as in PCR, bound ligated units dissociate from the marker and then serve as “target sequences” for ligation of excess probe pairs.

Following amplification, it may be desirable to separate the amplification product from the template and the excess primer for the purpose of determining whether specific amplification occurred. In some embodiments, amplification products are separated by agarose, agarose-acrylamide or polyacrylamide gel electrophoresis using standard methods (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). In some embodiments capillary electrophoresis or capillary gel electrophoresis may be used.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (see, e.g., Freifelder, D. Physical Biochemistry Applications to Biochemistry and Molecular Biology. 2nd ed. Wm. Freeman & Co., New York, N.Y. 1982; incorporated herein by reference in its entirety). In some embodiments, amplification product(s) are detected and/or quantified using mass spectrometry techniques.

Amplification products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In some embodiments, visualization is achieved indirectly. For example, following separation of amplification products, a nucleic acid probe is brought into contact with the amplified marker sequence. The probe preferably is conjugated to a chromophore but may be radiolabeled. In another embodiment, the probe is conjugated to a binding partner, such as an antibody or biotin, where the other member of the binding pair carries a detectable moiety. In some embodiments, detection is by Southern blotting and hybridization with a labeled probe. The techniques involved in Southern blotting are well known to those of skill in the art and can be found in many standard books on molecular protocols (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). Briefly, amplification products are separated by gel electrophoresis. The gel is then contacted with a membrane, such as nitrocellulose, permitting transfer of the nucleic acid and non-covalent binding. Subsequently, the membrane is incubated with a chromophore conjugated probe that is capable of hybridizing with a target amplification product. Detection is by exposure of the membrane to x-ray film or ion-emitting detection devices.

In some embodiments, all the basic essential materials and reagents required for detecting colorectal neoplasia associated with inflammatory bowel disease through detecting the methylation level (presence, absence, score, frequency) of markers specific for IBD-CRN (e.g., BMP3, vimentin, EYA4, NDRG4) in a stool sample obtained from the mammal are assembled together in a kit. Such kits generally comprise, for example, reagents useful, sufficient, or necessary for detecting and/or characterizing one or more markers specific for IBD-CRN (e.g., methylations in BMP3, vimentin, EYA4, NDRG4). In some embodiments, the kits contain enzymes suitable for amplifying nucleic acids including various polymerases, deoxynucleotides and buffers to provide the necessary reaction mixture for amplification. In some embodiments, the kits contain reagents necessary to perform real-time PCR. In some embodiments, the kits of the present invention include a means for containing the reagents in close confinement for commercial sale such as, e.g., injection or blow-molded plastic containers into which the desired reagent are retained. Other containers suitable for conducting certain steps of the disclosed methods also may be provided.

The present invention provides methods for monitoring a treatment of IBD-CRN. For example, in some embodiments, the methods may be performed immediately before, during and/or after a treatment to monitor treatment success. In some embodiments, the methods are performed at intervals on disease-free patients to ensure or monitor treatment success.

The present invention provides methods for obtaining a subject's risk profile for developing IBD-CRN. In some embodiments, the subject is diagnosed with IBD but not CRN. In some embodiments, such methods involve obtaining a stool sample from a subject (e.g., a human at risk for developing colorectal cancer; a human diagnosed with IBD but not CRN; a human undergoing a routine physical examination), detecting the presence or absence of one or more indicators specific for IBD-CRN (e.g., detecting the presence, absence, or level of markers specific for IBD-CRN in or associated with the stool sample (e.g., methylation level, score or frequency)) in the stool sample, and generating a risk profile for developing IBD-CRN based upon the detected presence, absence, or level of the indicators specific for IBD-CRN (e.g., BMP3, vimentin, EYA4, NDRG4). In some embodiments, the risk profile indicates a subject's risk for developing IBD-CRN or a subject's risk for re-developing IBD-CRN. In some embodiments, the risk profile indicates a subject to be, for example, a very low, a low, a moderate, a high, and a very high chance of developing or re-developing IBD-CRN. In some embodiments, a health care provider (e.g., an oncologist or gastroenterologist) will use such a risk profile in determining a course of treatment or intervention (e.g., colonoscopy, watchful waiting, referral to an oncologist, referral to a surgeon, etc.).

### EXAMPLES

The invention now being generally described, will be more readily understood by reference to the following example, which is included merely for purposes of illustration of certain aspects and embodiments of the present invention, and are not intended to limit the invention.

### Example I

This example describes the materials and methods for the experiments conducted during the course of developing embodiments for the present invention.

Tissue Study

Patients

Tissues were identified from a single-center archive of IBD-CRC cases and IBD control specimens after confirmation of histologic diagnosis. Cases and controls were matched for age (within a 10 year range), gender, disease duration, anatomic extent (left-sided vs. extensive) and PSC status (yes/no). DNA was extracted from paraffin-embedded tissues as described (see, e.g., Garrity-Park M M, Am J Gastroenterol 2008; 103:407-15; herein incorporated by reference in its entirety).

Mutation Marker Gene Sequencing

Candidate exons on APC, p53, K-ras, BRAF and PIK3CA were amplified in a real-time iCycler (BioRad, Hercules, Calif.) using real-time PCR reactions, performed with sense and antisense primers, IQ Supermix polymerase kit (BioRad) and 10 ng of genomic DNA. Products were run on a 2% agarose gel to confirm the presence of a single band and then cleaned with ExoSAP-IT (Affymetrix, Santa Clara, Calif.). The 14 exons of interest were bidirectionally sequenced on all 50 specimens on an ABI PRISM 3730×1 DNA analyzer (Applied Biosystems Inc, Foster City, Calif.). Sequences were screened for mutations using Mutation Surveyor (SoftGenetics, State College, Pa.) software and then compared to the National Center for Biotechnology Information database of single-nucleotide polymorphisms (dbSNP, http://, followed by, www.ncbi.nlm.nih., followed by, gov/projects/SNP/) to exclude common variants.

Real-Time Methylation-Specific PCR (MSP)

After bisulfite treatment, VIM, BMP3 and septin 9 PCR reactions for tissue DNA samples were performed with Taq polymerase (Invitrogen, Carlsbad, Calif.). During the course of the experiments, the laboratory protocol for MSP assays changed polymerase to SYBR Green master mix (Roche, Mannheim, Germany), which was used for EYA4 quantification. DNA was bisulfite treated using the EZ DNA Methylation Kit (Zymo Research, Orange, Calif.). Primers were designed to target the bisulfite-modified methylated sequences of gene promoters (IDT, Coralville, Iowa). The actin gene was quantified with real-time PCR using primers and probe recognizing bisulfite-converted sequence as a reference.

DNA Extraction

Using a modified Gentra (Gentra Systems Inc., Minneapolis, Minn.) protocol, DNA extracted from paraffin-embedded tissues was suspended in TE (10 mM Tris/0.1 mM EDTA, Integrated DNA Technologies, Coralville, Iowa). Quantification of total DNA was performed using the Picogreen assay (Invitrogen, Portland, Oreg.) (see, e.g., Garrity-Park M M, Am J Gastroenterol 2008; 103:407-15; herein incorporated by reference in its entirety).

Stool Study

Patients

Case patients with established IBD-CRN were recruited. Those who had undergone endoscopic or surgical treatment of neoplasia or with a history of other aerodigestive neoplasia were excluded. Each site recruited IBD control patients undergoing surveillance colonoscopy with an effort to match on age (in 5 year strata) and sex. After informed consent, participants were given a kit to collect stools prior to or at least one week after colonoscopy or sigmoidoscopy (see, e.g., Zou H, Cancer Epidemiol Biomarkers Prey 2006; 15:1115-9; Olson J, Diagn Mol Pathol 2005; 14:183-91 (see, e.g., each herein incorporated by reference in its entirety).

Sequence-Specific Gene Capture

A 2-gram equivalent of stool supernatant was used for multiplex capture of gene targets β-actin, EYA4, BMP3 and NDRG4) by amino conjugated oligonucleotides complementary to target sequences (see, e.g., Kisiel J B, Cancer 2011; herein incorporated by reference in its entirety). Stool samples were weighed and diluted 1:5 with additional buffer before incubation with polyvinylpyrrolidone (Crosby & Baker, Westport, Mass.) to remove PCR inhibitors. A 2-gram equivalent of stool supernatant was used for multiplex capture of 4 gene targets β-actin, VIM, EY44, BMP3 and NDRG4). Sodium chloride and guanidine thiocyanate (Sigma, St. Louis, Mo.) denaturation buffer were added to clarified stool supernatant and heated in a water bath before incubation and room temperature hybridization with carboxylic acid-coated capture beads with amino conjugated oligonucleotides complementary to target sequences (IDT). A 3-step wash in MOPS buffer was performed prior to heated tRNA buffer elution.

Assay of Methylated Markers

After capture, target DNA was bisulfite treated and quantitative allele-specific real-time target and signal amplification (QuARTS) reactions were performed on Roche 480 LightCyclers (Indianapolis, Ind.), as described (see, e.g., Ahlquist D A, Gastroenterology 2012; 142:248-56; herein incorporated by reference in its entirety). Quantitative allele-specific real-time target and signal amplification (QuARTS) reactions were performed on Roche 480 LightCyclers (Indianapolis, Ind.) using sets of primers, detection probes and invasive oligonucleotides (FAM, Hologic, Madison Wis.), fluorescence resonance energy transfers (FRETs), Cleavase 2.0 (Hologic), GoTaq DNA polymerase (Promega, Madison, Wis.), 10 mM MOPS, 7.5 mM MgCl₂, and 2500 μM of each dNTP for β-actin, mBMP3, mVIM and mNDRG4 genes. Bisulfite-treated CpGenome™ Universal methylated DNA (Millipore) and human genomic DNA (Novogen, Canada) were used as positive and negative controls. Each plate contained standards made of engineered plasmids, positive and negative controls, and water blanks. Standard curves were made of 10-fold serially diluted engineered plasmids with corresponding gene inserts to calculate the copy number of each marker based on an amplification efficiency of 1.95. EY44 methylation was assayed by methylation specific PCR, performed on a LightCycler 480 using SYBR Green I Master (Roche) as described (see, e.g., Kisiel J B, Cancer 2011; herein incorporated by reference in its entirety).

Stool Collection

Using a plastic bucket device mounted on the toilet seat, whole stools were collected and then stabilized with buffer solution and sealed with a water-tight lid. Upon laboratory receipt, stools were homogenized, aliquoted, and frozen at −80C until assayed (see, e.g., Zou H, Cancer Epidemiol Biomarkers Prey 2006; 15:1115-9; Olson J, Diagn Mol Pathol 2005; 14:183-91; each herein incorporated by reference in its entirety).

Statistical Analysis

Feasibility for IBD-CRN detection by stool DNA testing was defined a priori as sensitivity for neoplasia >50%. Based on conservative pre-study assumptions, it was estimated that 15 patients in the case group would provide 80% power to distinguish a true sensitivity of 70% from a null value of 40% with a 1-sided one sample proportion test at the 5% level. The distributions of each marker as a continuous variable were compared between cases and controls using the Wilcoxon rank sum test (JMP v8.0, SAS Institute, Cary N.C., USA). Logistic regression was used to calculate receiver operating characteristics (ROC) curves, from which specificity cut-offs were imputed and marker sensitivities (with 95% confidence intervals (CI)) were calculated. Multivariate logistic regression models assessed potential interaction and confounding by age, sex and clinical risk factors, including comorbid PSC (yes/no), disease duration (in years) and disease extent (left-sided/extensive).

### Example II

This example describes the results of the Tissue Study.

Clinical characteristics were well-matched between cases and controls (Table 1). There were no significant differences with the exception of inflammation score, which was higher in controls.

TABLE 1
Patient Characteristics for Tissue Study

| | Cases | Controls |
|---|---|---|
| | N = 25 | N = 25 |
| Male (%) | 16 (64) | 17 (68) |
| Mean age, years (SD) | 52 (14.4) | 50 (11.9) |
| Mean CUC duration, years (SD) | 20.7 (9.2) | 19.9 (8.3) |
| Extensive (%) | 21 (84) | 20 (80) |
| PSC (%) | 4 (16) | 3 (12) |
| Mean Inflammation score (SD)¹ | 0.17 (0.27) | 0.68 (0.66)² |

¹Using method of reference 26
²p = 0.001
SD, standard deviation
CUC, chronic ulcerative colitis
PSC, primary sclerosing cholangitis
Cases = Colorectal cancer in CUC,
Controls = CUC without neoplasia

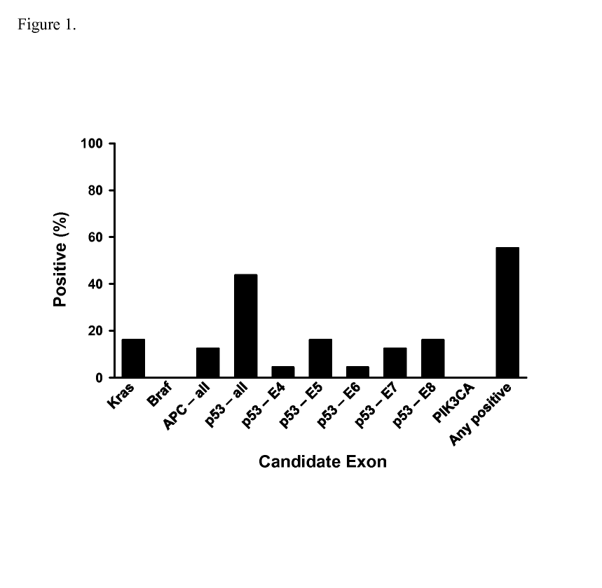

FIG. 1 summarizes the results of DNA sequencing for the case samples. Across 6 APC regions overlapping the mutation cluster region (1, 2, C, N, Y, L2), only 3 mutations were found. Four mutations were found on K-ras. As anticipated, p53 was the most informative marker with 11 mutations detected; however, these were spread out across a wide range of sites on all 5 tested exons. No mutations were identified on BRAF or PIK3CA. While specificity was 100% (no mutations found among control tissues), combined sensitivity for all 14 mutation markers was only 60%.

ROC curves were constructed for each of the methylation markers. Areas under the curve (AUC) were 0.97, 0.87, 0.81 and 0.73 for methylated EYA4 (mEYA4), VIM (mVIM), BMP3 (mBMP3) and Septin 9 respectively. Thus, mEYA4, mVIM and mBMP3 were selected for stool DNA testing in addition to methylated NDGR4 (mNDRG4), whose high discrimination for sporadic CRN was identified after the completion of the tissue study (see, e.g., Ahlquist D A, Gastroenterology 2012; 142:248-56; herein incorporated by reference in its entirety).

### Example III

This example describes the results of the Stool Study.

Given the excellent tissue discrimination observed with methylation markers in the tissue study, an analysis of stool from independent sets of cases and controls was performed. Nineteen IBD case patients with biopsy-confirmed CRN and 35 IBD control patients without CRN submitted stools (Table 2). Although IBD diagnoses and comorbid PSC were distributed evenly between the two groups, cases had significantly longer disease duration.

TABLE 2
Patient Characteristics for Stool Study

| | Cases | Controls |
|---|---|---|
| | N = 19 | N = 35 |
| CUC | 17 | 25 |
| Crohn's disease | 2 | 10 |
| % Male | 63 | 63 |
| Median age, years (range) | 60 (45-72) | 60 (45-77) |
| Median IBD duration, years (range) | 30 (2-50) | 14 (0-45)¹ |
| Extensive² | 17 | 19 |
| PSC (%) | 4 (21) | 5 (15) |

¹p = 0.0008
²Inflammation proximal to splenic flexure
CUC, chronic ulcerative colitis;
IBD, inflammatory bowel disease;
PSC, primary sclerosing cholangitis.
Cases = IBD with colorectal neoplasia,
Controls = IBD without neoplasia

Case neoplasms included 9 cancers with a median size of 2.3 cm (range 0.8-5 cm). Six of the 9 (67%) were proximal to the splenic flexure. Median stage (see, e.g., Edge SBB, D. R.; Compton, C. C.; Fritz, A. G.; Greene, F. L.; Trotti, A. (Eds.). AJCC Cancer Staging Manual. 7th ed: Springer, New York, 2010:646; herein incorporated by reference in its entirety) was I (range I to IIIC). Additional neoplasms included 8 discrete polypoid dysplastic lesions (3 high-grade dysplasia [HGD], 5 low-grade dysplasia [LGD]) with a median size of 2.3 cm (range 1.0-6.2) and two flat lesions (1 HGD, 1 LGD) detected on random biopsy (size unknown).

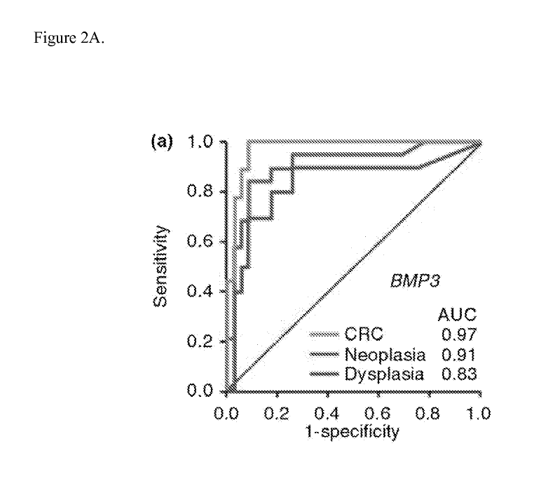

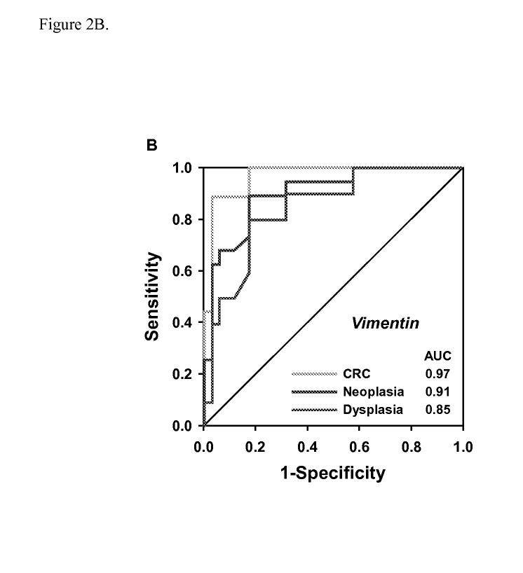

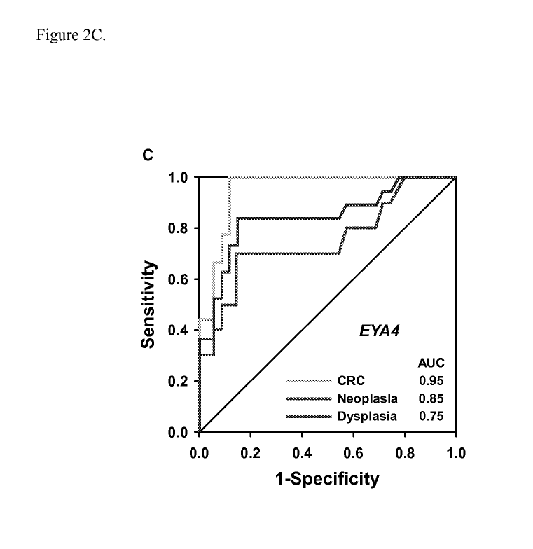

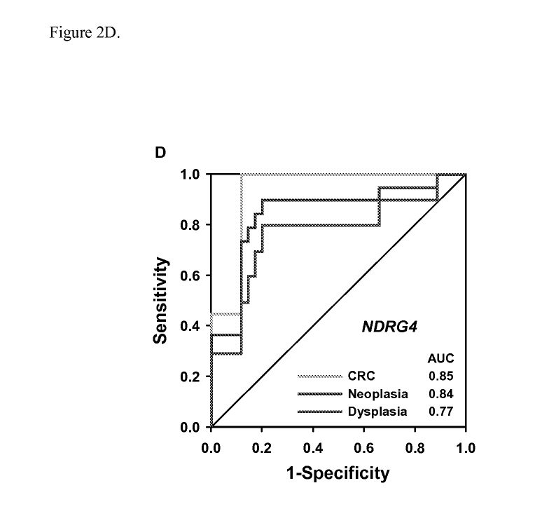

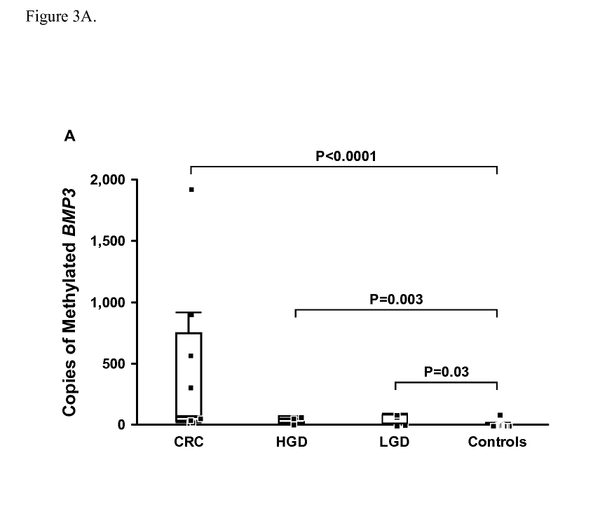

All 4 markers individually showed high discrimination for cancer (FIG. 2). AUCs with mBMP3, mVIM, mEYA4 and mNDRG4 were 0.97, 0.97, 0.95 and 0.94, respectively. For IBD-CRN the AUC with mBMP3, mVIM, mEYA4 and mNDRG4 were 0.91, 0.91, 0.85 and 0.84, respectively. For premalignant dysplasia, the AUC with mBMP3, mVIM, mEYA4 and mNDRG4 were 0.84, 0.85, 0.75 and 0.77, respectively. Stool assay of mBMP3 at 91% specificity was 100% sensitive for CRC and 84% sensitive for CRN (Table 3). At 89% specificity, mEYA4 and mNDRG4 each detected 100% of CRC and 74% of CRN. At 91% specificity, mBMP3 detected 70% of pre-malignant dysplasia. At 89% specificity, the combination of mBMP3 and mNDRG4 detected 100% of CRC, 89% of CRN, and 80% of premalignant dysplasia (100% of HGD, 67% of LGD). In multivariate analyses, methylation markers for CRN detection remained significant in models which included age, sex, extent of disease, or presence of PSC (Table 4). IBD duration was strongly correlated with marker levels in CRC but did not improve discrimination when modeled with stool DNA.

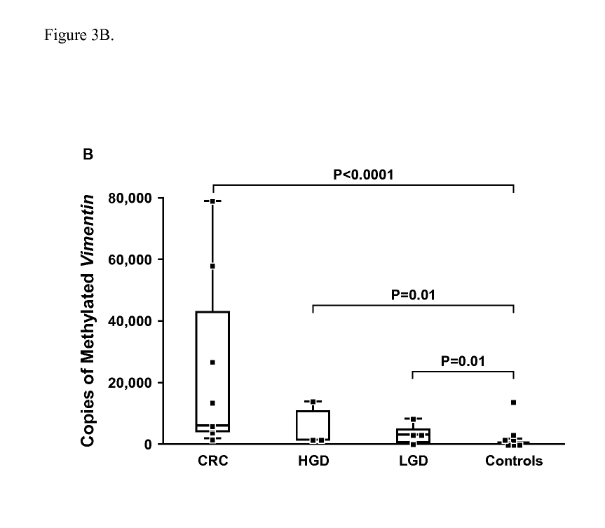

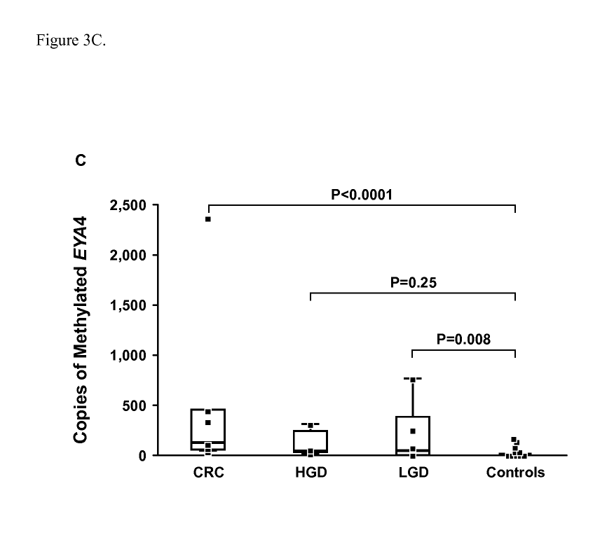

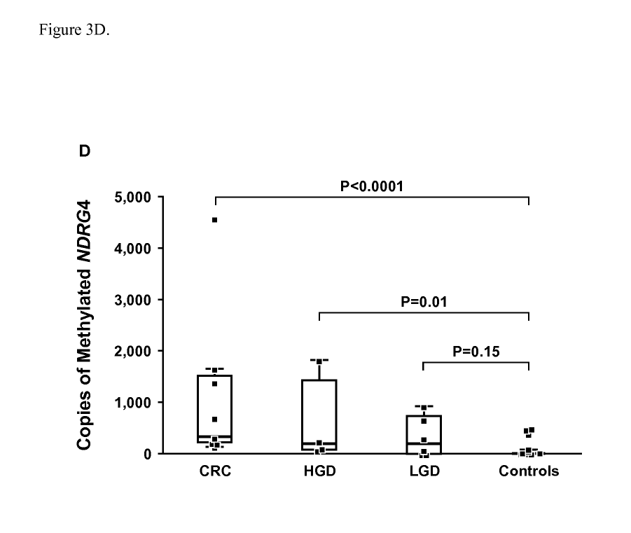

The dynamic range of methylated copy numbers between cases and controls was wide for each stool marker (FIG. 3). Among cases, copy numbers of mBMP3, mVIM, mEYA4 or mNDRG4 were not significantly different for proximal versus distal neoplasms (p=0.58, 0.73, 0.83 and 0.85, respectively).

TABLE 3
IBD-Associated Colorectal Neoplasm Detection Rates by Stool Assay of
Methylated DNA Markers

| | Sensitivity, % | | | |
|---|---|---|---|---|
| Specificity | (95% CI) | | | |
| Cut-off, % | mBMP3 | mVIM | mEYA4 | mNDRG4 |

CRC¹

| | | | | |
|---|---|---|---|---|
| 100 | 44 (15-77) | 44 (15-77) | 44 (15-77) | 44 (15-77) |
| 94 | 89 (51-99) | 89 (51-99) | 66 (31-91) | 44 (15-77) |
| 91 | 100 (63-100) | 89 (51-99) | 78 (40-96) | 44 (15-77) |
| 89 | 100 (63-100) | 89 (51-99) | 100 (63-100) | 100 (63-100) |

Neoplasia²

| | | | | |
|---|---|---|---|---|
| 100 | 21 (7-46) | 26 (10-51) | 37 (17-61) | 37 (17-61) |
| 94 | 68 (43-86) | 68 (43-86) | 53 (29-74) | 37 (17-61) |
| 91 | 84 (60-96) | 68 (43-86) | 63 (39-82) | 37 (17-61) |
| 89 | 84 (60-96) | 68 (43-86) | 74 (48-90) | 74 (48-90) |

Dysplasia

| | | | | |
|---|---|---|---|---|
| 100 | 0 (NA) | 10 (5-46) | 10 (5-46) | 30 (8-65) |
| 94 | 50 (20-80) | 50 (20-80) | 40 (14-73) | 30 (8-65) |
| 91 | 70 (35-91) | 50 (20-80) | 50 (20-80) | 30 (8-65) |
| 89 | 70 (35-91) | 50 (20-80) | 50 (20-80) | 50 (20-80) |

¹CRC = colorectal cancer
²Neoplasia = CRC + premalignant dysplasia combined
NA, could not be calculated

TABLE 4
Results of Multivariate Models of Association between Clinical Endpoints
and Methylation Markers Assayed from Stool, Adjusting for Clinical
Variables

| | P-values by endpoint | | |
|---|---|---|---|
| Model | Cancer⁵ | Neoplasia | Dysplasia |
| mBMP3 | 0.04 | 0.004 | 0.01 |
| Age¹ | 0.98 | 0.87 | 0.61 |
| Age × mBMP3 | 0.86 | 0.92 | 0.91 |
| mBMP3 | 0.009 | 0.004 | 0.03 |
| Sex | 0.03 | 0.11 | 0.31 |
| Sex × mBMP3 | 0.07 | 0.09 | 0.33 |
| mBMP3 | 0.45 | 0.006 | 0.01 |
| IBD Duration² | 0.05 | 0.47 | 0.12 |
| IBD Duration × mBMP3 | 0.07 | 0.85 | 0.67 |
| mBMP3 | 0.02 | 0.02 | 0.06 |
| IBD Extent³ | Unstable | 0.03 | 0.09 |
| IBD Extent × mBMP3 | Unstable | 0.08 | 0.25 |
| mBMP3 | 0.03 | 0.25 | 0.26 |
| PSC⁴ | Unstable | 0.36 | 0.28 |
| PSC × mBMP3 | Unstable | 0.36 | 0.35 |
| mVIM | 0.01 | 0.01 | 0.05 |
| Age | 0.37 | 0.96 | 0.97 |
| Age × mVIM | 0.50 | 0.94 | 0.93 |
| mVIM | 0.02 | 0.002 | 0.02 |
| Sex | 0.03 | 0.03 | 0.33 |
| Sex × mVIM | Unstable | 0.02 | 0.08 |
| mVIM | 0.67 | 0.02 | 0.04 |
| IBD Duration | 0.12 | 0.20 | 0.85 |
| IBD Duration × mVIM | 0.34 | 0.87 | 0.30 |
| mVIM | 0.03 | 0.002 | 0.06 |
| IBD Extent | Unstable | 0.001 | 0.08 |
| IBD Extent × mVIM | Unstable | 0.01 | 0.08 |
| mVIM | 0.04 | 0.33 | 0.35 |
| PSC | 0.98 | 0.44 | 0.38 |
| PSC × mVIM | Unstable | 0.44 | 0.41 |
| mEYA4 | 0.02 | 0.01 | 0.02 |
| Age | 0.84 | 0.37 | 0.95 |
| Age × mEYA4 | 0.93 | 0.24 | 0.20 |
| mEYA4 | 0.01 | 0.003 | 0.03 |
| Sex | 0.38 | 0.48 | 0.48 |
| Sex × mEYA4 | Unstable | 0.57 | 0.57 |
| mEYA4 | 0.19 | 0.008 | 0.03 |
| IBD Duration | 0.12 | 0.12 | 0.13 |
| IBD Duration × mEYA4 | 0.49 | 0.80 | 0.59 |
| mEYA4 | 0.02 | 0.01 | 0.14 |
| IBD Extent | 0.38 | 0.04 | 0.11 |
| IBD Extent × mEYA4 | Unstable | 0.55 | 0.90 |
| mEYA4 | 0.01 | 0.07 | 0.12 |
| PSC | Unstable | 0.92 | 0.72 |
| PSC × mEYA4 | Unstable | 0.98 | 0.73 |
| mNDRG4 | 0.03 | 0.008 | 0.02 |
| Age | 0.82 | 0.47 | 0.96 |
| Age × mNDRG4 | 0.17 | 0.07 | 0.14 |
| mNDRG4 | 0.003 | 0.003 | 0.02 |
| Sex | 0.11 | 0.52 | 0.41 |
| Sex × mNDRG4 | Unstable | 0.39 | 0.62 |
| mNDRG4 | 0.67 | 0.01 | 0.03 |
| IBD Duration | 0.08 | 0.03 | 0.07 |
| IBD Duration × mNDRG4 | 0.18 | 0.56 | 0.95 |
| mNDRG4 | 0.01 | 0.01 | 0.30 |
| IBD Extent | 0.36 | 0.03 | 0.13 |
| IBD Extent × mNDRG4 | Unstable | 0.52 | 0.69 |
| mNDRG4 | 0.01 | 0.06 | 0.12 |
| PSC | 0.98 | 0.83 | 0.57 |
| PSC × mNDRG4 | Unstable | 0.77 | 0.52 |

1. Age in years at time of study consent
2. Years since inflammatory bowel disease (IBD) diagnosis
3. Left-sided colitis versus colitis proximal to splenic flexure
4. Presence or absence of comorbid primary sclerosing cholangitis (PSC)
5. Multivariate regression models with unstable terms were repeated, excluding the unstable variable(s)

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A method for detecting colorectal neoplasia in a subject having inflammatory bowel disease, comprising:
a) obtaining DNA from an excreted stool sample of said subject;
b) determining the level of one or more nucleic acid polymer markers having altered methylation in said DNA from said excreted stool sample, wherein said one or more nucleic acid polymer markers having altered methylation are specific for colorectal neoplasia associated with inflammatory bowel disease.

2. The method of claim 1, wherein said one or more nucleic acid polymers with altered methylation comprises a region selected from the group consisting of a CpG island and a CpG island shore.

3. The method of claim 2, wherein said CpG island or shore is present in a coding region or a regulatory region of a gene selected from the group consisting of BMP3, vimentin, NDRG4, and EYA4.

4. The method of claim 2, wherein said determining of the level of altered methylation of a nucleic acid polymer comprises determining the methylation score of said CpG island or island shore.

5. The method of claim 2, wherein said determining of the level of altered methylation of a nucleic acid polymer comprises determining the methylation frequency of said CpG island or island shore.

6. The method of claim 1, wherein said determining of the level of a nucleic acid polymer with altered methylation is achieved by a technique selected from the group consisting of methylation-specific PCR, quantitative methylation-specific PCR, methylation-sensitive DNA restriction enzyme analysis, quantitative bisulfite pyrosequencing, and bisulfite genomic sequencing PCR.

7. The method of claim 1, further comprising: c) generating a risk profile using the results of steps a) and b).

8. The method of claim 1, wherein said colorectal neoplasm is premalignant.

9. The method of claim 1, wherein said colorectal neoplasm is malignant.

10. The method of claim 1, wherein said inflammatory bowel disease is selected from the group consisting of ulcerative colitis and Crohn's disease.

11. A kit for detecting the presence of a colorectal neoplasm in a mammal having inflammatory bowel disease, said kit comprising reagents useful, sufficient, or necessary for detecting and/or characterizing one or more nucleic acid polymers with altered methylation specific for colorectal neoplasm associated with inflammatory bowel disease from a stool sample, wherein said one or more nucleic acid polymers are selected from the group consisting of BMP3, vimentin, NDRG4, and EYA4.

12. The kit of claim 11, wherein said one or more nucleic acid polymers with altered methylation comprises a region selected from the group consisting of a CpG island and a CpG island shore.

13. The kit of claim 12, wherein said CpG island or shore is present in a coding region or a regulatory region.

14. The kit of claim 12, wherein said determining of the level of altered methylation of a nucleic acid polymer comprises determining the methylation score of said CpG island or island shore.

15. The kit of claim 12, wherein said determining of the level of altered methylation of a nucleic acid polymer comprises determining the methylation frequency of said CpG island or island shore.

16. The kit of claim 11, wherein said determining of the level of a nucleic acid polymer with altered methylation is achieved by a technique selected from the group consisting of methylation-specific PCR, quantitative methylation-specific PCR, methylation-sensitive DNA restriction enzyme analysis, quantitative bisulfite pyrosequencing, and bisulfite genomic sequencing PCR.

17. The kit of claim 11, wherein said colorectal neoplasm is premalignant.

18. The kit of claim 11, wherein said colorectal neoplasm is malignant.

19. The kit of claim 11, wherein said inflammatory bowel disease is selected from the group consisting of ulcerative colitis and Crohn's disease.
